(19) United States
(12) Patent Application Publication
(10) Pub. No.: US 2009/0011456 A1
(43) Pub. Date: Jan. 8, 2009

(54) Methods for the Diagnosis and Prognosis of Graft Versus Host Disease By Measurement of Peripheral Cd3+Cd4+Cd8Beta+ Cells

(75) Inventors: Clayton Smith, Vancouver (CA); Ryan Brinkman, Coquitlam (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: BRITISH COLUMBIA CANCER AGENCY BRANCH, Vancouver, BC (CA)

(21) Appl. No.: 12/281,675

(22) PCT Filed: Mar. 12, 2007
(86) PCT No.: PCT/CA2007/000389

§ 371 Date: Sep. 4, 2008

Related U.S. Application Data
(60) Provisional application No. 60/780,877, filed on Mar. 10, 2006.

(51) Int. Cl. C12Q 1/02

(52) U.S. Cl. 435/29

(57) ABSTRACT

Acute graft versus host disease (GvHD) is one of the most significant clinical problems in allogeneic blood and marrow transplantation. Currently, there is no unequivocal diagnostic test for GvHD until the disease is well developed and can be recognized histologically. The invention provides a blood based test for diagnosis and/or prognosis of GvHD, allowing assessment of risk for developing GvHD prior to appearance of clinical symptoms. Using flow cytometry, peripheral blood mononuclear cells are assessed for an increase in proportion and fluctuation of CD3⁺CD4⁺CD8β⁺ cells. An increase in presence or proportion, or high fluctuation in this cell population prior to onset of clinically recognized indicators of GvHD is predictive of later development of GvHD.

### CROSS REFERENCE TO RELATED APPLICATIONS

This application claims the benefit of priority of U.S. Provisional Patent Application No. 60/780,877 filed Mar. 10, 2006, which is incorporated herein by reference in its entirety.

### FIELD OF THE INVENTION

The present invention relates generally to diagnostic and/or prognostic method for graft versus host disease.

### BACKGROUND OF THE INVENTION

Acute graft-versus-host disease (GvHD) occurs in allogeneic hematopoietic stem cell transplant (SCT) recipients when donor immune cells in the graft initiate an attack on the skin, gut, liver and other tissues of the recipient (1-6). The pathophysiology of GvHD is currently felt to occur through several phases (2, 4, 6). In the first phase, damage by the chemotherapy or radiotherapy used in the transplant preparatory regimen causes host tissues to secrete inflammatory cytokines. This results in activation of alloreactive donor T-cells that recognize HLA and minor histocompatability antigen disparities on host cells. Subsequently, the donor T cells and other immune effectors elaborate a variety of inflammatory cytokines including TNF-α, IFN-γ, IL-13, IL-5 and others resulting in the widespread tissue damage observed clinically. A variety of other immune cells, including dendritic cells, B-cells, natural killer cells and macrophages may also play important roles in GvHD through additional mechanisms (7-11).

It is likely that the outcome of GvHD could be improved if it were treated as early as possible in a pre-emptive fashion, before the full-blown clinical syndrome develops. In addition, if the diagnosis of GvHD could be made more definitively, only those patients who absolutely required steroids and other immunosuppressive medications would be treated. Currently however, there is no definitive method for detecting GvHD during its preclinical stage or to unequivocally distinguish GvHD from infections or drug toxicities unless there are advanced histologic features on biopsy samples. For these reasons, it is important to develop rapid, reliable and accurate tests for the prediction and diagnosis of GvHD.

The development of microarray technologies has ushered in a new era of diagnostic tests (12). Attempts have been made to use microarrays to identify gene expression patterns in peripheral blood leukocytes that would be diagnostic of GvHD (13, 14). However, gene microarray data provides averaged gene expression information from peripheral blood leukocyte populations that are actually quite heterogeneous in nature. Consequently, the averaging effects of microarray analysis may miss important variations in expression of individual genes within different subsets of cells. In addition, contributions from small populations of immune cells that may be important to the development of GvHD could be missed altogether.

Flow cytometry (FCM) offers a potential alternative to gene microarray analysis for rapidly defining complex changes in heterogeneous populations (15-17). Multiple dyes, lasers and detectors can be used to simultaneously collect multiple fluorescence emission signatures from cell populations representing as little as 0.1% of the total sample (18). Thousands of fluorescently conjugated antibodies and fluorescent dyes are now commercially available to provide a wide array of cellular measurements including cell phenotype, intracellular cytokine expression, cell cycle status and recently, signal transduction pathway activation. Recently, high throughput or high content FCM systems that can analyze thousands of individual samples per day have been developed, which can provide rich data sets on various types of cells (19, 20).

Managing the large amounts of data generated by high-throughput/high content flow cytometry system is a significant challenge. When analyzing parameters such as changes in immune effector cell populations over time that could contribute to GvHD, the temporal aspect of the data introduces an additional level of complexity as well. GvHD can occur several days to several months after allogeneic transplant and data analysis needs to account for this variation within a study population. Recently, several algorithms treating time as a continuous variable have been introduced to deal with this issue, including spline-based methods such as linear, cubic and B-splines (21-29). Splines are mathematical representations of smooth curves that pass through two or more points. Using splines to model time series data can avoid problems such as data overfitting, and the methodology is well-suited for the analysis of small numbers of data points (22, 30).

There is no methodology currently available for detecting onset or prognosis of GvHD. Great benefit to individuals and society could be derived from a blood-based test capable of determining likelihood of onset or potential progression of GvHD in individuals prior to onset, allowing medical resources to be deployed to appropriate individuals prior to progression of GvHD.

### SUMMARY OF THE INVENTION

It is an object of the present invention to provide a method of diagnosing or determining the prognosis of graft-versus-host disease.

In a first embodiment, the present invention provides a method for diagnosing graft versus host disease (GvHD) or determining risk of developing GvHD in a susceptible subject comprising: measuring CD3⁺CD4⁺CD8β⁺ cells in peripheral blood, and comparing CD3⁺CD4⁺CD8β⁺ cells with a control value; wherein an increase in CD3⁺CD4⁺CD8β⁺ cells in peripheral blood, a high level of CD3⁺CD4⁺CD8β⁺ cells in peripheral blood, or high fluctuation in CD3⁺CD4⁺CD8β⁺ cells in peripheral blood is indicative of GvHD or a risk of developing GvHD.

In a further embodiment, there is provided a method of treating graft versus host disease (GvHD) comprising diagnosing GvHD or risk of developing GvHD in a susceptible subject according to the method described herein, and providing to a subject diagnosed with or at risk of developing GvHD an anti-GvHD therapeutic regime prior to observing clinical symptoms of GvHD.

In further embodiment, the present invention provides a kit for diagnosing graft versus host disease (GvHD) or determining risk of developing GvHD in a susceptible subject comprising antibodies for detection of CD3⁺CD4⁺CD8β⁺ cells via flow cytometry analysis, and instructions for use.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1 is a schematic representation of a method according to an embodiment of the invention.

FIG. 1B is a schematic representation of a method according to a further embodiment of the invention.

FIG. 2 is a representative FC-HCS analysis of PBMCs from patients with GvHD.

FIG. 3A shows the first step in data analysis pipeline leading to the invention: data extraction and transformation from high content flow cytometry analysis.

FIG. 3B shows the second step in the data analysis pipeline leading to the invention: temporal classification.

FIG. 3C depicts data obtained in the data analysis pipeline outlined in FIGS. 3A and 3B, including determining population proportion of the target cell type and charting the proportion and signal of target cell type post-transplant.

FIG. 4 shows the pattern of CD3⁺CD4⁺CD8β⁺ cells following allogeneic transplant differentiates between patients with and without GvHD.

FIG. 5 is a representative gating strategy for CD3⁺CD4⁺CD8β⁺ cells and co-expression of CD4, CD8β and CD8 on these cells in a patient with GvHD.

FIG. 6 illustrates a representative flow cytometric analysis of CD3⁺CD4⁺CD8β⁺ cells in patients who developed GvHD compared to patients who did not develop GvHD.

### DETAILED DESCRIPTION

Generally, the present invention provides a method for diagnosis and/or prognosis of graft-versus-host disease.

Acute graft versus host disease (GvHD) is diagnosed by clinical and histologic criteria that are often non-specific and typically apparent only after the disease is well established. GvHD is mediated by donor T-cells and other immune effector cells. The instant invention allows for diagnosis and prognosis of graft versus host disease based on changes in peripheral blood lymphocyte populations that have been found to be predictive of GvHD. To arrive at the instant invention, a wide array of peripheral blood lymphocyte populations were assessed from subjects susceptible to developing GvHD, and a specific cell population was found to be predictive of the development of GvHD.

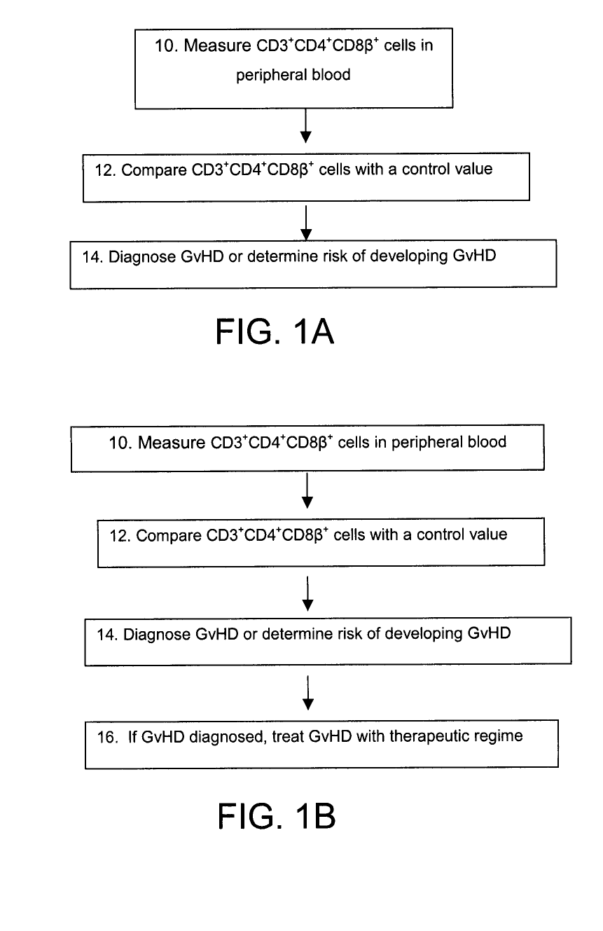

An embodiment of the invention relates to a method for diagnosing graft versus host disease (GvHD) or determining risk of developing GvHD in a susceptible subject. This embodiment is depicted in FIG. 1A. The method comprises (10) measurement of CD3⁺CD4⁺CD8β⁺ cells in peripheral blood, and (12) comparison of CD3⁺CD4⁺CD8β⁺ cells with a control value. An increase in CD3⁺CD4⁺CD8β⁺ cells in peripheral blood, a high level of CD3⁺CD4⁺CD8β⁺ cells in peripheral blood, or high fluctuation in CD3⁺CD4⁺CD8β⁺ cells in peripheral blood can be viewed as indicative of GvHD or a risk of developing GvHD. Thus, GvHD can be diagnosed or the risk of developing GvHD can be determined (14). Prognosis can also be determined, as the likely severity of GvHD may be determined prior to onset of clinical symptoms.

Susceptible subjects may be those having received an allogeneic blood and/or marrow transplant, a tissue or organ graft or transplant, or any combination of these. While it is most likely that graft versus host disease may occur in an acute format during a time period from 14 to 90 days post-transplantation, the method of the invention can be used to make a diagnosis or determination of risk during time periods outside of this range, for example, from immediately following transplantation up until a year or more post-transplant.

The method of diagnosis or determining risk may involve periodic measurement of CD3⁺CD4⁺CD8β⁺ cells in peripheral blood. Blood samples may be taken from subjects prior to transplantation (which may, for example be used as a control values), and following transplant on a regular or periodic schedule. Periodic measurements may be conducted at regular intervals, depending on the progress of the subject, and the preference of the health care provider, for example, the interval for repeating samples may be daily, if a subject is viewed as very likely to be at risk. Less frequent intervals may be a more practical approach to periodic sampling in some health care settings, and may be desirable in some instances, for example blood samples may be analysed every 10 days, or each month following transplantation. An exemplary interval for sampling is weekly, and this may be particularly beneficial in the 14 to 90 days post-transplantation when GvHD is most likely to occur.

The measurement of CD3⁺CD4⁺CD8β⁺ cells in peripheral blood can be assessed in any variety of ways to allow comparison with a control value. For example, the measurement of such cells may be expressed as a percentage of total peripheral blood cells or lymphocytes, as a ratio against another cell population that remains relatively constant, or as an absolute concentration or value, such as number of cells per microlitre of blood, serum or plasma analysed.

The comparison with a control value may be conducted in a variety of ways. For example, if a subject has had a previous measurement taken, his or her current measurement of CD3⁺CD4⁺CD8β⁺ cells can be compared with the previous measurement as a baseline. This type of a comparison may address individual variation among subjects, and would allow for expression of comparison with a control as a percentage increase or an absolute increase. Comparison with an individual's previous measurements also allows for detection of high fluctuation or variability in CD3⁺CD4⁺CD8β⁺ cell levels, high fluctuation also being correlated with diagnosis of and/or risk of developing GvHD. The degree of fluctuation most indicative of risk of development can be observed in a larger population, so that a health care provider can review a subject's previous values and determine whether the fluctuation in values is above a certain threshold of fluctuation. An individual's previous measurement can be one determined prior to transplant, and/or in a previous assessment interval.

The control value against which a subject's CD3⁺CD4⁺CD8β⁺ cell measurement can be compared may be assessed against a threshold increase determined in a larger population. For example, in a larger population, it may be determined that a 5%, 10%, 15%, 20%, or 25% increase, on a week over week basis is indicative of diagnosis or risk developing GvHD, then as soon as this level of increase is found, a health care provider may decide that treatment of GvHD is warranted, even in the absence of clinical symptoms or a definitive biopsy result. Alternatively, an absolute increase, using units such as number of cells per microlitre, could be assessed based on an individual's own previous levels.

The control value to be used for comparison can be selected from among age-matched or sex-matched populations. Further, the analysis of blood may vary from centre to centre, and thus certain health care centres may wish to use internally-derived control values to reduce variability attributable to differences in equipment or techniques between other centres. Other factors can also be matched to allow for suitable control selection, such as days post-transplant, the type of transplant conducted, etc., which can readily be determined by those skilled in the field.

A threshold control value may be determined from a healthy population, from among a population of transplant recipients without GvHD, or from a population of transplant recipients observed to subsequently develop GvHD, based on review of values immediately leading to development of clinical symptoms.

The invention relates to the measurement of cells in peripheral blood having all three surface markers: CD3⁺CD4⁺ and CD8β⁺. Such an observation, that this suite of markers found on one cell (CD3⁺CD4⁺CD8β⁺) can serve as a basis for diagnosis or determination of risk, had not heretofore been made or conceived of. Diagnosis of acute and/or chronic GvHD can be made on the basis of this suite of markers.

There are various ways in which cells simultaneously possessing these three surface markers can be identified and quantified. While certain conventional assays of surface markers may indeed permit detection of each of the three surface markers in this suite of markers, such assays may be unable to determine whether an individual peripheral blood cell has all three markers simultaneously. The invention was made based on a powerful statistical analysis capable of uncovering correlations that are non-linear in nature, in combination with high content (or high-throughput) flow cytometry. Flow cytometry is one tool that can be used to quantify cells having all of these surface markers. However, other methods may be used. Immunohistochemistry or staining of cells with antibodies can be used, provided the markers can be detected on the same cells. For example, a three stain overlay on a slide may be combined with automated image recognition. Further, cells may be captured using antibodies coupled to iron particles and subsequently magnetically isolated. Isolation could be conducted either in a positive selection manner to enrich for the cells, or in a negative manner to subtract unwanted cells. The isolated cells could then be analysed by western blot or immunohistochemistry.

While the invention was made possible through the use of high powered statistical methodology in combination with high content flow cytometry, neither the use of the same statistical analysis using spline analysis as described herein below, nor the use of high content flow cytometry is required in the application or reduction to practice of the invention in a clinical setting. Now that the relationship between CD3⁺CD4⁺CD8β⁺ peripheral blood cells and diagnosis/risk of GvHD has been established, a health care provider can use a simple comparison (either an increase or a threshold value) to make an appropriate determination. Further, because the appropriate cell type has now been identified, regular flow cytometry machines set with appropriate gating can be used, and a high content or high-throughput machine is not required, making this method accessible in health care settings where high-end and expensive state of the art flow cytometry equipment may not yet be available. Advantageously, this renders the method of the instant invention accessible to almost any health care setting having access to flow cytometry.

An embodiment of the invention may additionally comprise assessment of one or more parameters relating to immune function, which can be used in combination with the measurement of CD3⁺CD4⁺CD8β⁺ cells in order to diagnose or determine risk of developing GvHD. Such parameters relating to immune function can include any one or combination of the following: cytokine production, intracellular cytokines, measurement of T-regulatory cells, apoptosis status, cell cycle status, cellular activation phenotypes or signal transduction phenotypes. By combining such measurements, an accurate and individualized profile of immune function, or the failure thereof, can be developed and monitored over time to permit diagnosis or determination of the prognosis for GvHD.

According to an embodiment of the invention, for example, as depicted in FIG. 1B, there is provided a method of treating graft versus host disease (GvHD) comprising determining GvHD or risk of developing GvHD in a susceptible subject according to the method described above, as for example depicted in FIG. 1A, and on the basis of the determination or diagnosis made, providing to the subject at risk of developing GvHD an anti-GvHD therapeutic regime (16) prior to observing clinical symptoms of GvHD. Such a therapeutic regime could comprise administration of immunosuppressive medication, such as steroids (for example methylprednisone or prednisone), cyclosporine, steroids in combination with cyclosporine, monoclonal antibodies such as anti-CD3, -CD5, and -IL-2 antibodies, Mycophenolate Mofetil™, Alemtuzumab™ (Campath), Antithymocyte globulin (ATG), FK506™, Sirolimus™, or any other therapeutic regime, including a combination therapy, that may be determined appropriate by a health care provider. Advantageously, this method may allow early treatment of GvHD, strategically used in those individuals at the highest risk of developing GvHD. Early treatment can be of great benefit to the long-term prognosis or progression of the condition. Further for those individuals determined not to be at risk of developing GvHD on the basis of their blood test result can avoid side-effects associated with the therapeutic regime by avoiding unnecessary treatment.

Another aspect of the invention provides a kit for diagnosing graft versus host disease (GvHD) or determining risk of developing GvHD in a susceptible subject. Such a kit may comprise antibodies for detection of CD3⁺CD4⁺CD8β⁺ cells via flow cytometry analysis, along with instructions for use. Such a kit may provide instructions for determining an appropriate interval of periodic analysis, or may direct the user to appropriate calculations for comparison with a control, or threshold values based on large population studies, should a user prefer to us a population-based control. The antibodies to be provided with the kit can be any of those used in the experimental example described hereinbelow, or other antibodies as would be known or available to those of skill in the art.

The powerful statistical and analytical combination of bioinformatics/spline analysis with high content flow cytometry has permitted the analysis that lead to the instant invention. This combination can permit identification of other cell types using surface markers to diagnose or assess risk of other medical conditions that have not been previously detectable prior to onset of clinical symptoms or biopsy. Such conditions may be either related or unrelated to the field of organ transplantation.

Further, other transplant-related complications and conditions may also be rendered predictable and thus preventable on the basis of the CD3⁺CD4⁺CD8β⁺ cell population identified. For example, conditions such as organ transplant rejection, susceptibility to infection post-transplantation, also common problems post-transplantation may be diagnosed and/or prevented on the basis of the instant invention.

Embodiments of the present invention permit diagnosis of a patient at risk of developing GvHD by using flow cytometry to determine the proportion of CD3⁺CD4⁺CD8β⁺ cells in a peripheral blood sample. An increase in the proportion or an increase in fluctuation of this population of cells over time can identify a patient who will proceed to develop GvHD.

In an embodiment the invention, there is a method incorporating the following steps to identify patients who will proceed to develop full blown GvHD.

Flow cytometry is used to capture quantitative data on cell populations in a blood sample from a patient. Using the appropriate gating, the flow cytometer will identify CD3⁺CD4⁺CD8β⁺ cells. Cell staining techniques and the parameters for gating of the flow cytometer are performed using standard methods know to individuals skilled in the art of flow cytometric analysis

The data is transformed to express the cell lineages and their abundance as a percentage of total peripheral blood mononuclear cells. Data can be expressed in any acceptable format, such as the following three related formats: proportion data; absolute concentration by normalization to total blood count; or as the Log2 ratio of change relative to the value at the time of transplantation by a logarithmic transformation.

A dynamic profile can be calculated and visualized graphically by treating time as a continuous variable and processing data acquired in discrete values using algorithms such as splines that will generate data in the format of a continuous curve. A appropriate spline may be linear, cubic or a B-splines. However, transformation of data is not required. Data obtained in the previous step can be compared to an appropriate standard, or compared to a patient's own previous data.

Optionally, should bioinformatics or spline analysis be used, a preprocessing step can be utilized, which may comprise smoothing by roughness penalty and/or a spline imputation process resulting in an output of smoothed curves. Again, such analysis is not required for individual data, but is useful in processing large quantities of data, for example, in determining a control value based on population data.

Diagnosis of a patient can be performed as described previously by comparison with a standard. Further, a classifier built on a reference data set may be used. Such a classifier can diagnose a patient by identifying features in a temporal model built, for example, by Functional Linear Discriminant Analysis. A temporal model can be built using samples from a reference set of patients with and without GvHD. The data from such a reference set of patients can be processed as described above.

In a further embodiment of the invention, additional cell surface markers may used in the flow cytometry process. Such additional cell surface markers can be included in the classifier built on the reference data set and can be used in the process of diagnosing patients at risk for GvHD. Such additional cell surface markers for hematopoietic cells can be selected by those skilled in the art of immunology.

A further embodiment of the invention may include assessment of additional parameters of immune function in order to diagnose GvHD. Such additional parameters include cytokine production, intracellular cytokines, measuring T-regulatory cells, apoptosis status, cell cycle status, cellular activation phenotypes and signal transduction phenotypes known to those skilled in the art of immunology. These data, when combined with the assessment of the CD3⁺CD4⁺CD8β⁺ cell population can also permit diagnosis of GvHD, and may provide additional information about the diagnosis or prognosis of the disease.

An exemplary use of the invention is to employ weekly monitoring of a patient's status. Flow cytometry is used to capture quantitative data on cell populations in a blood sample from the patient to identify CD3⁺CD4⁺CD8β⁺ cells. The data obtained is expressed in terms of cell population abundance, either as a concentration or as a percentage of total peripheral blood mononuclear cells. A dynamic profile for an individual patient can be observed through weekly monitoring. Should a patient's CD3⁺CD4⁺CD8β⁺ cells show high fluctuation, or surpass a certain threshold relative to a control value, a diagnosis can be made.

### EXPERIMENTAL

Using high content flow cytometry and bioinformatics/spline analysis, the following experimental approach and results were used to arrive at the instant invention. It is to be understood that in order to practice the instant invention, neither a repeat of the bioinformatics analysis described herein, nor the use of high content flow cytometry equipment (as opposed to standard flow cytometry equipment) is required.

Peripheral blood samples from 31 patients undergoing allogeneic blood and marrow transplant were analyzed for the proportion of 121 different subpopulations defined by 4-color combinations of lymphocyte phenotypic and activation markers at progressive time points post-transplant. Samples were processed using a newly developed high content flow cytometry technique and subjected to a spline- and FLDA-based temporal analysis technique. This strategy identified a consistent post-transplant increase in the proportion and extent of fluctuation of CD3⁺CD4⁺CD8β⁺ cells in patients who developed GvHD compared to those that did not. While larger prospective clinical studies will be necessary to validate these results, this study demonstrates that high content flow cytometry coupled with temporal analysis is a powerful approach for developing new diagnostic tools and may be useful for developing a sensitive and specific predictive test for GvHD. Based on developments in bioinformatics and flow cytometry, the instant invention has been made possible. The invention advantageously combines the advantages of high content flow cytometry with the power of modern bioinformatics to determine if there are patterns of cells in the peripheral blood that correlate with a variety of physiologic or disease states including GvHD. Using the high content FCM and the spline-based analysis methods outlined above in a pilot study of 31 patients undergoing allogeneic transplantation, we analyzed whether increases or decreases in the proportion of one or more of 121 different peripheral blood leukocyte populations predicted the subsequent development of acute GvHD. Of these populations, an increase in the proportion of CD3⁺CD4⁺CD8β⁺ cells 7-21 days post transplant best correlated with the subsequent development of acute GvHD. These findings suggest that this population should be studied further for its possible biologic role in GvHD and that larger prospective clinical studies should be conducted to validate its predictive accuracy. In addition, this work demonstrates that high content FCM and spline-based analysis is a promising approach to developing diagnostic tools for GvHD and other processes.

Materials and Methods

Study Patients. Thirty-one patients undergoing HLA matched sibling and unrelated donor allogeneic blood and marrow transplantation were enrolled at the Moffitt Cancer Center. Of the 31 patients enrolled in this study, 21 patients were diagnosed with acute GvHD while 3 patients did not develop either acute or chronic graft versus host disease and thus were used as controls. The remaining patients were not included in the analysis either because they i) died prior to 100 days post transplant and it could not be determined if they would have subsequently developed GvHD or not (n=2), ii) developed de-novo chronic graft versus host disease which may have confounded the analysis for acute GvHD (n=3), iii) were lost to follow-up (n=J) or iv) had insufficient clinical samples collected (n=1). The details of the patient demographics, stem cell source, transplant related treatments and acute GvHD are summarized in Table 1. The diagnosis and grading of GvHD was made using previously published criteria (31).

TABLE 1
Summary of Patient Information

| | | | Acute | Acute | |
|---|---|---|---|---|---|
| Patient | Conditioning | | GvHD | GvHD | |
| No. | Regimen | Donor | Day | Grade | Clinical Outcome |
| 1 | CyTBI | MUD | 26 | III | |
| 2 | CBV | SIB | | none | |
| 3 | CyTBI | MUD | 23 | IV | died d61 |
| 4 | CBV | SIB | | none | died d278 |
| 5 | CyTBI | SIB | 59 | III | |
| 6 | BuCy | SIB | 19 | III | |
| 7 | NMT | SIB | 39 | III | died d89, GvHD |
| 8 | BuCy | SIB | | none | de novo cGvHD; |
| | | | | | d122 |
| 9 | CBV | SIB | 43 | III | |
| 10 | CyTBI | MUD | 11 | I | |
| 11 | BuCy | SIB | 68 | I | |
| 12 | BuCy | SIB | 22 | III | |
| 13 | BuCy | SIB | 48 | III | |
| 14 | BuCyTBI | MUD | 28 | II | |
| 15 | CBV | SIB | 19 | II | |
| 16 | CyTBI | MUD | 10 | II | died, d74 |
| 17 | BuCy | SIB | | none | |
| 18 | CBV | SIB | | n/a | died d54 |
| 19 | NMT | SIB | 77 | II | |
| 20 | BuCy | SIB | | n/a | died d55 |
| 21 | CyTBI | MUD | 54 | III | |
| 22 | BuCy | SIB | 32 | III | |
| 23 | BuCy | SIB | 22 | III | |
| 24 | BuCy | SIB | 37 | III | |
| 25 | NMT | SIB | 44 | I | died d89 |
| 26 | CBV | SIB | | none | de novo cGvHD |
| 27 | NMT | SIB | 31 | II | |
| 28 | CyTBI | MUD | 51 | I | |
| 29 | | SIB | | n/a | lost to follow-up |
| 30 | | SIB | | none | Chronic GvHD |
| 31 | BuCy | SIB | | none | died d109, |
| | | | | | disease relapse |

Isolation of peripheral blood mononuclear cells. Samples of peripheral blood were collected using RB approved processes into EDTA containing tubes pre-transplant and then weekly for at least 100 days post transplant. Peripheral blood mononuclear cells (PBMCs) were isolated using Ficoll-Hypaque and then were cryopreserved for subsequent batch analysis. At the time of analysis, cells were thawed and aliquoted into 96 well plates at 1×10⁴ to 1×10⁵ cells per well in IMDM/10% FCS. The 96 well plates were then stained and analyzed as described below.

Flow cytometric high content screening. The 96 well plates were stained with 10 different 4-color antibody combinations as described in Table 2.

TABLE 2
Markers used in high content flow cytometric analysis

| | Ab-FITC | Ab-PE | Ab-PerCP | Ab-APC | |
|---|---|---|---|---|---|
| Panel | (clone) | (clone) | (clone) | (clone) | Detects |
| 1 | CD15 | CD45 | CD14 | CD33 | Leukocytes |
| | (MMA) | (J.33) | (MfP9) | (P67.6) | |
| 2 | CD4 | CD8β | CD3 | CD8 | T helper/T |
| | (SK3) | (2ST8.5H7) | (SK7) | (SK1) | suppressors |
| 3 | CD16 | CD2 | CD3 | CD56 | NK cells |
| | (NKP15) | (S5.2) | (SK7) | (B159) | |
| 4 | CD10 | CD20 | CD19 | CD22 | B-cells |
| | (HI10a) | (L27) | (SJ25C1) | (S-HCL-1) | |
| 5 | TCRαβ | TCRγδ | CD3 | CD5 | γδ+/αβ+ |
| | (WT31) | (11F2) | (SK7) | (L17F12) | T-cells |
| 6 | CD44 | CD25 | CD3 | CD69 | Activated |
| | (L178) | (2A3) | (SK7) | (L78) | T-cells |
| 7 | CD4 | CD134 | CD3 | CD8 | Activated |
| | (SK3) | (ACT35) | (SK7) | (SK1) | T-cells |
| 8 | CD4 | CD122 | CD3 | CD8 | Activated |
| | (SK3) | (TU27) | (SK7) | (SK1) | T-cells |
| 9 | CD45RA | CD45RO | CD3 | CD4 | Memory/ |
| | (L48) | (UCHL1) | (SK7) | (SK3) | naïve T-cells |
| 10 | CD45RA | CD45RO | CD3 | CD8 | Memory/ |
| | (L48) | (UCHL1) | (SK7) | (SK1) | naïve T-cells |

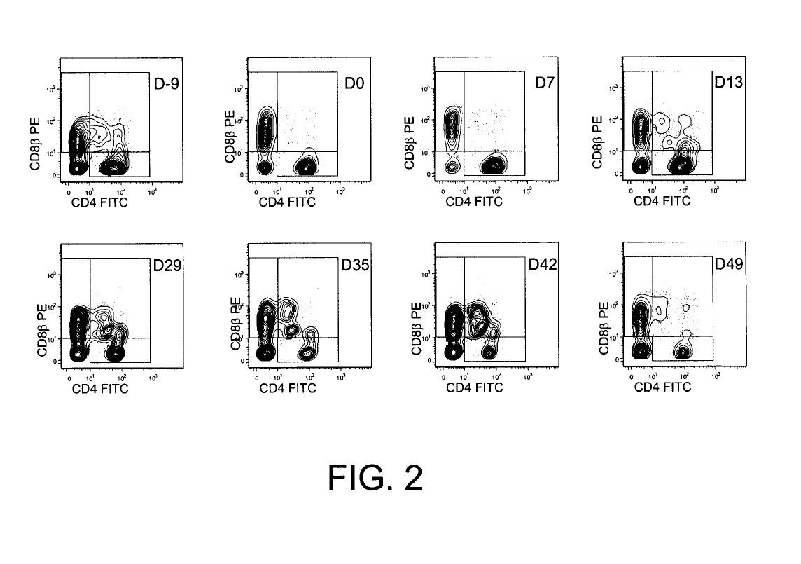

All antibodies were obtained from BD Biosciences except anti-CD45-PE and anti-CD8β-PE, which were from Immunotech. All staining and flow cytometric analysis was done using a flow cytometric high content screening (FC-HCS) technique described previously (19). Briefly, in the FC-HCS technique, all staining and analysis procedures were miniaturized so that small numbers of cells could be stained in 96 well plates with optimally diluted fluorescently conjugated antibodies. All staining procedures were performed by a Biomek® 2000 robotic fluid handler (Beckman Instruments, Schaumburg, Ill.) using a series of mini-programs developed with BioWorks™ software (Beckman Instruments). Cell washings were performed either by centrifugation or by vacuum aspiration through a filter bottom plate (Silent Screen Plate, Nunc™, Rochester N.Y.), using a vacuum manifold (96-Filtration System-Beckman Coulter) controlled by the Bioworks software. Flow cytometric analysis was performed on a FACSCalibur device equipped with a 488 nm argon laser and a ˜635 nm red dye laser (Becton Dickinson (BD), San Jose, Calif.). Samples of cells were delivered directly from 96 well plates to the FACSCalibur using prototypes of a Microtiter Well Plate device (BD). The BD Multiwell Plate Manager/Multiwell Autosampler (MPM/MAS) software (BD) was used for the collection and annotation of data. A typical example of the primary flow cytometric data collected in this fashion is depicted in FIG. 2.

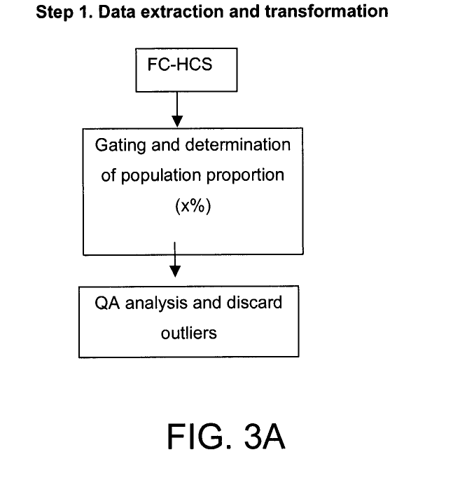

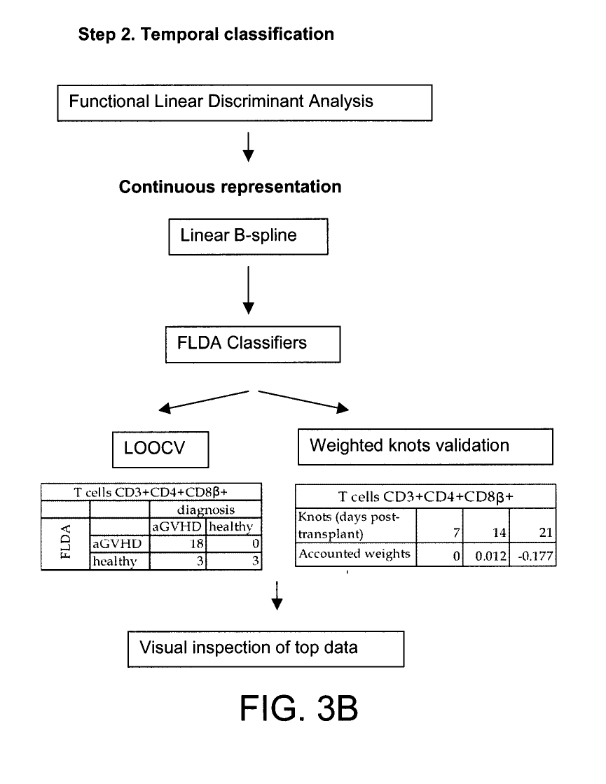

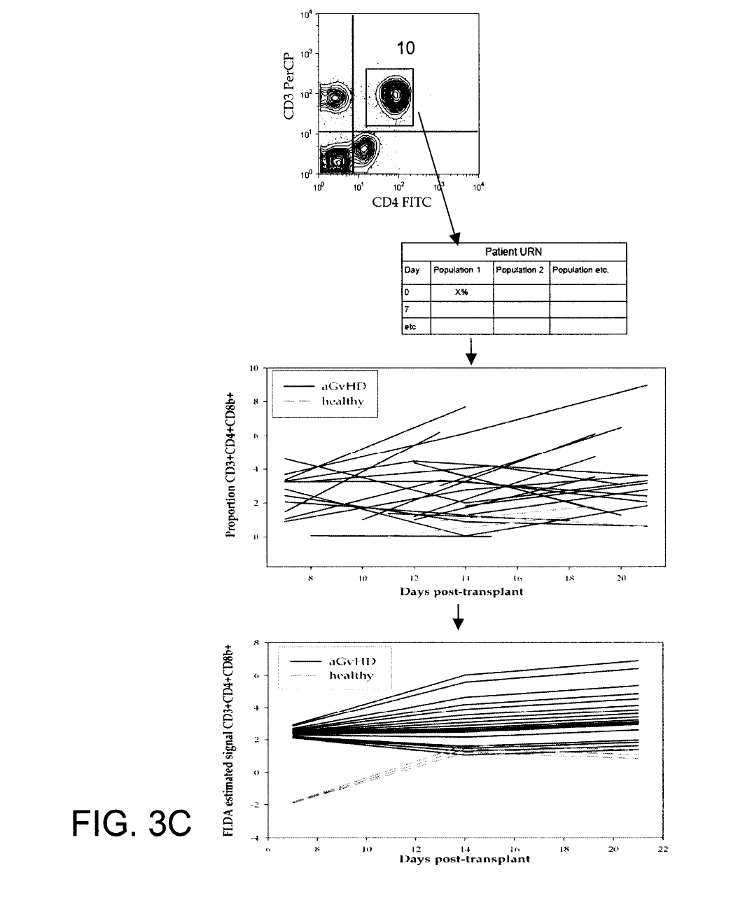

FIG. 2 shows representative FC-HCS analysis of PBMCs from patients with GvHD. Each panel depicts 2-D contour diagrams of 4-color analyses from a single patient at progressive time points following transplant (day −9 to day +49) using anti-CD4 and anti-CD8β. Typical gates for defining the various subpopulations are outlined. Statistical analysis. The strategy for analysis of the FC-HCS data is summarized in FIGS. 3A and 3B. Data obtained using the steps shown in FIGS. 3A and 3B are shown in FIG. 3C.

FIGS. 3A and 3B shows a summary of the data analysis pipeline. The overall strategy for performing temporal analysis of the high content flow cytometry analysis is summarized. FC-HCS=High Content Flow Cytometry, FLDA=Functional Linear Discriminant Analysis, LOOCV=Leave One Out Cross Validation. The details of each of these steps is described in the Methods section. FIG. 3A shows the data extraction and transformation step 1. FIG. 3B shows the temporal classification of step 2. FIG. 3C shows depicts data obtained in the data analysis pipeline outlined in FIGS. 3A and 3B, including determining population proportion of the target cell type and charting the proportion and signal of target cell type post-transplant.

Batch analysis of the FCS files was performed using FlowJo Software (TreeStar, Palo Alto, Calif.) to determine the proportion of each gated population within peripheral blood mononuclear cells. A total of 121 distinct cell populations were defined in the 10 four-color staining panels using gates on two-dimensional contour plots as depicted in the representative example in FIG. 2. This data was then exported as text files to generate tabular data. Data from samples taken between 7 to 21 days post-transplant were selected to develop the predictive test since we sought to develop a test that would be useful 1-2 weeks prior to the onset of GvHD. Patient data was separated into two groups representing either affected (developed acute GvHD) or unaffected (developed neither acute nor chronic GvHD) patients. Splines were fit to the time series proportion data for each cell type for each patient. Functional Linear Discriminant Analysis (FLDA) was then employed to generate an average curve for each group, based on a signal plus noise model as previously described (32). FLDA was performed in MATLAB (Natick, Mass.) based on linear basis B-splines with three vertices (i.e., one for each week). All the primary flow cytometric data from measurements with high estimated sensitivity and specificity were inspected visually to confirm the spline based data analysis strategy. Lastly, the leave-one out validation (LOOV) technique was used to estimate the error of the FLDA classifier.

Results

To determine if a cellular signature of acute GvHD could be defined in the weeks prior to the onset of the full-blown clinical syndrome, PBMCs from weekly blood samples were collected starting immediately after allogeneic transplant through −day +100. These samples were stained using 10 panels of 4-color antibody combinations (Table 2) and flow cytometry data was collected using the FC-HCS technique. Gating was performed on different populations within each stained sample to generate a total of 121 populations for subsequent statistical analysis. An example of a representative 4-color, 6-parameter staining combination and gated subpopulations is depicted in FIG. 2. The data was then analyzed as outlined in FIGS. 3A and 3B for any changes in cell populations occurring between 7 and 21 days post-transplant that correlated with the development of acute GvHD, which in this series of patients occurred on average 35 days (±17 days) post-transplant. Data obtained from the steps conducted in FIGS. 3A and 3B are shown in FIG. 3C.

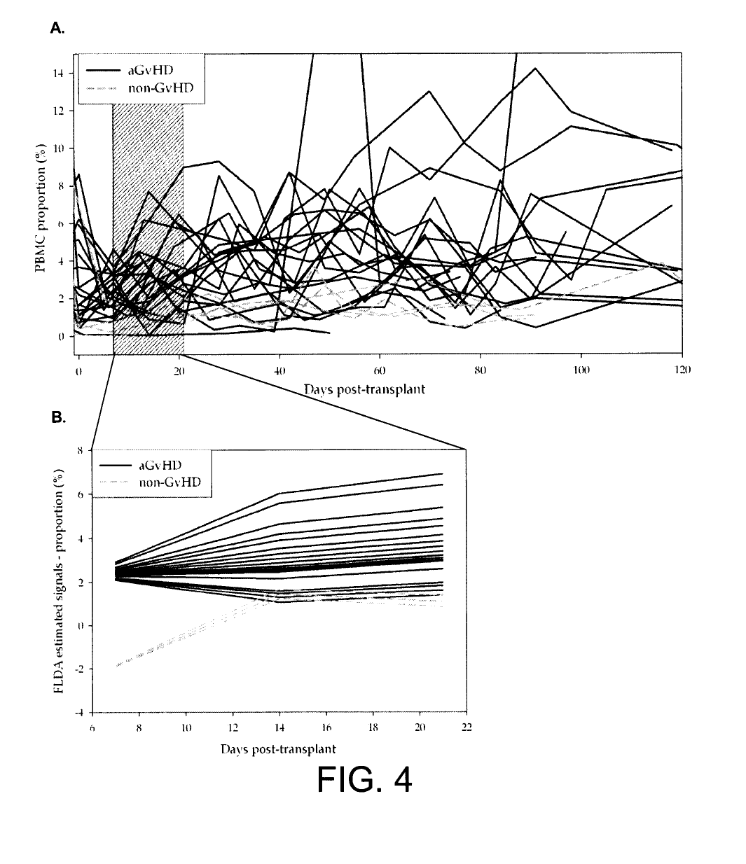

Of the 121 populations analyzed in this fashion, a population of lymphocytes that co-stained with antibodies directed against CD3, CD4 and CD8β (CD3⁺CD4⁺CD8β⁺ cells) was found to have the highest correlation with the development of acute GvHD (FIG. 4, panels A and B, as well as in data not shown).

FIG. 4 illustrates the pattern of CD3⁺CD4⁺CD8β⁺ cells following allogeneic transplant differentiates between patients with and without GvHD. Transplant recipients who did not develop GvHD (dashed lines) showed little variation in the proportion of CD3⁺CD4⁺CD8β⁺ cells while patients who later developed GvHD (solid lines) tended to have significantly higher and more variable values within the first 7-21 days post-transplant. Results are shown for both raw data (Panel A) and FLDA estimated true signal (Panel B).

Patients who did not develop acute GvHD had little variation in the proportion of CD3⁺CD4⁺CD8β⁺ cells in the 7-21 days following transplant, while acute GvHD patients had a higher and more variable proportion of CD3⁺CD4⁺CD8β⁺ cells within this time period (FIG. 4). To confirm whether these findings from the spline-based analysis and FLDA reflected actual changes in the flow cytometric analysis of the patient's peripheral blood lymphocytes rather than an anomaly arising from the analysis strategy, the original flow data for the CD3⁺CD4⁺CD8β⁺ cells was inspected manually for each patient at each time point.

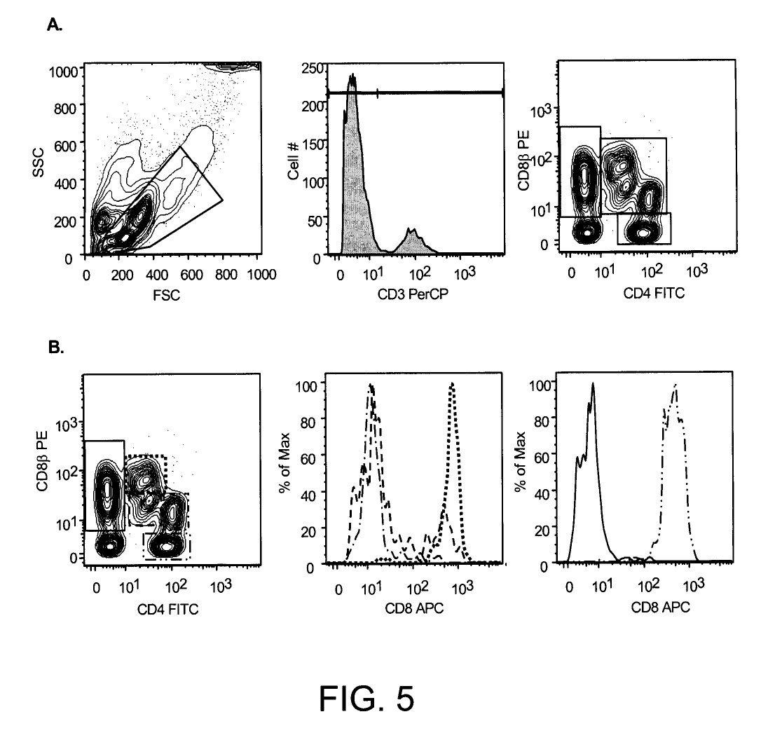

FIG. 5 shows a representative gating strategy for CD3⁺CD4⁺CD8β⁺ cells and co-expression of CD4, CD8β and CD8 on these cells in a patient with GvHD. In panel A, an example of the CD3⁺CD4⁺CD8β⁺ gating strategy is depicted. Cells were initially gated through a lymphocyte/mononuclear cell gate defined by FSC and SSC properties in the left hand contour plot, then through a CD3⁺ gate (middle histogram) and finally through the upper right gate in the CD4 vs. CD8β contour plot. In panel B, the co-expression of CD4, CD8β and CD8 is demonstrated for various sub-populations defined by the various colored gates in the left hand CD4 vs. CD8β contour plot. The middle histogram demonstrates CD8 expression on CD4^{dim}CD8β^{br (}dotted line), CD4^{dim}CD8β^{dim} (dashed line) and CD4^{br}CD8β^{dirn} (dash/dot line) subpopulations and the right hand histogram demonstrates CD8 expression on the CD8β⁺CD4− (solid line) and CD4⁺CD8− (dash/dot/dot line) subpopulations defined in the left hand contour plot.

The gating strategy for defining the CD3⁺CD4⁺CD8β⁺ population detected in the spline analysis is presented in FIG. 5, panel A. The entire CD3⁺CD4⁺CD8β⁺ population in many patients consisted of 2-3 distinct subpopulations defined by varying levels of staining with antibodies directed against CD4, CD8β and CD8 (FIG. 5, panel B). While these populations could not be clearly distinguished in all patients at all time points, a subpopulation which co-expressed high levels of both CD8β and CD8 (CD3⁺CD4⁺CD8β^{br}CD8⁺ cells) could typically be detected.

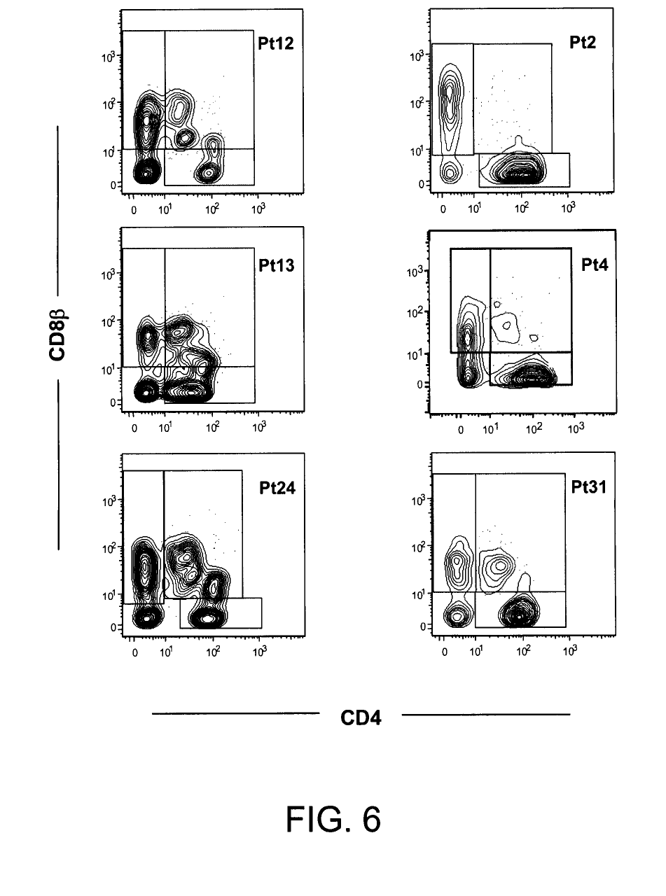

FIG. 6 shows a representative flow cytometric analysis of CD3⁺CD4⁺CD8β⁺ cells in patients who developed GvHD compared to patients who did not develop GvHD. The dot plots are from three representative patients 1 week prior to the diagnosis of GvHD for the patients who developed GvHD and on D+35 (the average day of GvHD development) in the 3 controls who did not develop GvHD.

As depicted in FIG. 6 (and from data not shown), a visually distinct difference was noted for the proportion of CD3⁺CD4⁺CD8β⁺ and CD3⁺CD4⁺CD8β^{br}CD8⁺ cells between the patients who developed GvHD and those who did not, supporting the temporal data analysis strategy. A leave-one out classifier built on these observations correctly predicted the absence of GvHD with 100% specificity and 86% sensitivity when based on the entire CD3⁺CD4⁺CD8β⁺ population (Table 3). Most of the predictive power of this population resided in the CD3⁺CD4⁺CD8β^{br}CD8⁺ cells as opposed to other populations seen in this four color staining panel that did not co-stain with one or more of the antibodies to CD4, CD8 or CD8β (FIG. 5, panel B and Table 3).

TABLE 3
Leave one out validation (LOOV) of the predictive power of
CD3⁺CD4⁺CD8β⁺ and other populations with the development acute
GvHD

| | All patients | Grade II-IV | Grade III/IV |
|---|---|---|---|
| | w/GvHD | GvHD | GvHD |
| Cell population | Sensitivity/Specificity (%) | | |
| CD3⁺CD4⁺CD8β⁺ | 86/100 | 92/100 | 83/100 |
| CD3⁺CD8β⁺CD4⁺CD8⁺ | 71/100 | 83/100 | 76/100 |
| CD3⁺CD8β^{dim}CD8⁻ | 90/0 | 82/67 | 83/67 |
| CD3⁺CD8β⁺CD4⁻ | 81/33 | 76/33 | 75/33 |
| CD3⁺CD8⁺CD8β⁻ | 81/33 | 76/33 | 75/33 |
| CD3⁺CD4⁺CD8β⁻ | 90/33 | 100/33 | 100/0 |
| CD3⁺ | 90/33 | 94/33 | 92/33 |

Discussion

In the current study, we hypothesized that a sensitive and specific flow cytometric cellular signature for acute GvHD could be developed based on analyzing a wide array of peripheral blood cell populations for phenotypic and activation markers using high content flow cytometry and temporal based statistical tools. We found that the FC-HCS technique could be readily applied to analyzing a large clinical sample set and that it efficiently yielded a robust data set for subsequent analysis. As we have reported previously, the FC-HCS technique was able to process 500-1000 samples per day with excellent flow cytometric staining and analysis profiles (for example, see FIG. 2) (19). While other high throughput flow cytometry approaches are more efficient, FC-HCS is well suited for detecting small populations of cells in different samples using multiparameter analysis since there is almost no carryover between individual samples that could cause false positive results (data not shown) (20). Since the development of acute GvHD occurred at different time points, it was important to use temporal analysis tools (e.g. spline- and ELDA based methods) to look for statistically meaningful differences in the various leukocyte populations between patients diagnosed with GvHD and the controls and to build a predictive model. This approach may also be suitable for other projects where clinical outcomes occur at variable time points.

In this study, the FC-HCS technique coupled with the temporal analysis determined that elevations in the proportion of CD3⁺CD4⁺CD8β⁺ cells within the first 7-21 days post-transplant predicted the development of GvHD with the highest sensitivity and specificity of any of the tested populations. A subset of these cells that also co-stained with an anti-CD8 antibody (i.e. CD3⁺CD4⁺CD8β^{br}CD8⁺ cells) also predicted the development of GvHD with a high sensitivity and specificity whereas other populations which lacked co-expression of either CD4, CD8 or CD8β had relatively low predictive value (Table 3). To conclusively determine whether the CD3⁺CD4⁺CD8β⁺ or CD3⁺CD4⁺CD8β^{br}CD8⁺ cell value is a reliable predictor of GvHD, a larger prospective clinical study will need to be conducted which is sufficiently powered to yield statistically stronger results than obtained in this initial pilot study. It will be important in this future study to control for reactivation of CMV and development of other infectious diseases as well as additional clinical events to ensure that the flow cytometric signature is specific to GvHD. In addition, other antibody panels should be incorporated to determine if cell populations that were not looked for in this initial pilot study, including T-regulatory cells and others, have greater sensitivity and specificity an the CD3⁺CD4⁺CD8β⁺ population identified in this study (33, 34).

Ultimately, these data illustrate that a simple weekly blood test can be used wherein multi-color staining and flow cytometric analysis are performed to predict the development of GvHD. This test can allow for pre-emptive treatment of GvHD, similar to the pre-emptive strategies used for CMV reactivation following allogeneic transplantation (35).

Subsequent clinical trials could then test whether pre-emptive treatment with steroids or other immunosuppressive agents could improve the outcome compared to traditional treatments that are initiated only after the clinical syndrome is established. Other uses of this test could include determining how much immunosuppressive treatment should be initiated, when treatment failure has occurred and as a guide for tapering immunosuppressive therapy. A similar approach may also be applicable to developing a test for chronic GvHD or other transplant related outcomes.

If the CD3⁺CD4⁺CD8β⁺ or CD3⁺CD4⁺CD8β^{br}CD8⁺ cell populations identified in this report are validated as accurate and reliable predictors of GvHD in future studies, it will also be important to further clarify their biology and relevance to the clinical manifestations of GvHD. This cell type appears to be a T-cell subset that co-expresses CD4, CD8αβ heterodimers (detected by the 2ST8.5H7 antibody, Table 2) and CD8aa homodimers (detected by the SKI antibody, Table 1). One type of CD4⁺CD8⁺ (double positive) T-cell is found in the thymus as an intermediate stage of T-cell development. These can be found at low levels in the peripheral blood of healthy individuals and at higher levels during viral infection and other conditions where they are thought to represent premature egress of immature thymic T-cells (36-39). However, the CD3⁺CD4⁺CD8β⁺ CD8⁺ population detected in patients after allogeneic transplant in our study appears to express lower levels of CD4 than typical intra-thymic double positive T-cells, and may represent a different cell type altogether (40). This possibility is supported by reports of double positive T-cells in the lamina propria of rhesus macaques and in mice following epicutaneous immunization that have a variety of phenotypic and functional properties that suggest they are mature T-cells rather than premature release of immature thymic T-cells (41, 42). Similarly, in humans, CD4^{dim}CD8⁺ T-cells have been observed in healthy blood donors, HIV infected persons and kidney transplant recipients that also appear distinct from intrathymic immature double positive T-cells (43). CD4⁺CD8⁺ T-cells have been found in the blood of both HCV infected persons and normal controls that have properties consistent with differentiated effector memory cells as well (36). Together, these observations suggest that the double positive T-cells we have observed in the peripheral blood of persons following allogeneic transplant may be mature antigen specific cells. It will be particularly interesting in future studies to characterize the CD3⁺CD4⁺CD8β⁺ CD8⁺ population for a variety of properties including activation and differentiation status, expression of tissue and lymph node homing markers, the pattern of intracellular cytokine secretion, TCR Vβ repertoire diversity and antigen responsiveness as previously described in virally infected persons (36). In addition, if a similar population were observed in murine models of allogeneic transplant, more direct studies of its relevance to GvHD could be performed (44).

In the preceding description, for purposes of explanation, numerous details are set forth in order to provide a thorough understanding of the embodiments of the invention. However, it will be apparent to one skilled in the art that these specific details are not required in order to practice the invention.

The above-described embodiments of the invention are intended to be examples only. Alterations, modifications and variations can be effected to the particular embodiments by those of skill in the art without departing from the scope of the invention, which is defined solely by the claims appended hereto. All documents referred to herein are incorporated herein by reference in their entirety.

### REFERENCES

1. Chao N J. Graft-versus-host disease: the viewpoint from the donor T cell. Biology of Blood and Marrow Transplantation 1997; 3:1-10.
2. Couriel D, Caldera H, Champlin R, Komanduri K. Acute graft-versus-host disease: pathophysiology, clinical manifestations, and management. Cancer 2004; 101:1936-1946.
3. Gilliam A C. Update on Graft versus Host Disease. Progress in Dermatology 2004; 23:251-257.
4. Goker H, Haznedaroglu I C, Chao N J. Acute graft-vs-host disease: pathobiology and management. Experimental Hematology 2001; 29:259-277.
5. Martin P J, Schoch G, Fisher L, et al. A retrospective analysis of therapy for acute graft-versus-host disease: initial treatment. Blood 1990; 76:1464-1472.
6. Reddy P. Pathophysiology of acute graft-versus-host disease. Hematological Oncology 2003; 21:149-161.
7. Vakkila J, Thomson A W, Hovi L, Vettenranta K, Saarinen-Pihkala U M. Circulating dendritic cell subset levels after allogeneic stem cell transplantation in children correlate with time post transplant and severity of acute graft-versus-host disease. Bone Marrow Transplant 2005; 35:501-507.
8. Chan G W, Gorgun G, Miller K B, Foss F M. Persistence of host dendritic cells after transplantation is associated with graft-versus-host disease. Biol Blood Marrow Transplant 2003; 9: 170-176.
9. Haraguchi K, Takahashi T, Matsumoto A, et al. Host-residual invariant NK T cells attenuate graft-versus-host immunity. J Immunol 2005; 175:1320-1328.
10. Nakamura A, Kobayashi E, Takai T. Exacerbated graft-versus-host disease in Pirb−/− mice. Nat Immunol 2004; 5:623-629.
11. Lo J W, Leung A Y, Huang X R, et al. Macrophage migratory inhibitory factor (MIF) expression in acute graft-versus-host disease (GVHD) in allogeneic hemopoietic stem cell transplant recipients. Bone Marrow Transplant 2002; 30:375-380.
12. Margalit 0, Somech R, Amariglio N, Rechavi G. Microarray-based gene expression profiling of hematologic malignancies: basic concepts and clinical applications. Blood Rev 2005; 19:223-234.
13. Akilesh 5, Shaffer D J, Roopenian D. Customized molecular phenotyping by quantitative gene expression and pattern recognition analysis. Genome Res 2003; 13:1719-1727.
14. Ichiba T, Teshima T, Kuick R, et al. Early changes in gene expression profiles of hepatic GVHD uncovered by oligonucleotide microarrays. Blood 2003; 102:763-771.
15. Srour E F, Yoder M C. Flow cytometric analysis of hematopoietic development. Methods Mol Med 2005; 105:65-80.
16. Dunphy C H. Applications of flow cytometry and immunohistochemistry to diagnostic hematopathology. Arch Pathol Lab Med 2004; 128:1004-1022.
17. Goolsby C. A look at where we came from and where we are going. Cytometry 2004; 58A:53-54.
18. Owen R G, Rawstron A C. Minimal residual disease monitoring in multiple myeloma: flow cytometry is the method of choice. British Journal of Haematology 200S; 128:732-733.
19. Gasparetto M, Gentry T, Sebti S, et al. Identification of compounds that enhance the anti-lymphoma activity of rituximab using flow cytometric high-content screening. J Immunol Methods 2004; 292:59-71.
20. Edwards B S, Oprea T, Prossnitz E R, Sklar L A. Flow cytometry for high-throughput, high-content screening. Curr Opin Chem Biol 2004; 8:392-398.
21. Bar-Joseph Z, Gerber G, Simon I, Gifford D K, Jaakkola T S. Comparing the continuous representation of time-series expression profiles to identify differentially expressed genes. Proceedings of the National Academy of Sciences of the United States of America 2003; 100:10146-10151.
22. Bar-Joseph Z, Gerber G K, Gifford D K, Jaakkola T S, Simon I. Continuous representations of time-series gene expression data. Journal of computational biology: a journal of computational molecular cell biology 2003; 10:341-356.
23. Dc Hoon M J, Imoto S, Miyano S. Statistical analysis of a small set of time-ordered gene expression data using linear splines. Bioinformatics (Oxford, England) 2002; 18: 1477-1485.
24. Irizarry R A, Tankersley C, Frank R, Flanders S. Assessing homeostasis through circadian patterns. Biometrics 2001; 57:1228-1237.
25. James G, Hastie T. Functional Linear Discriminant Analysis for Irregularly Sampled Curves. Journal of the Royal Statistical Society: Series B 2001; 63:533-550.
26. James G, Sugar C. Clustering for Sparsely Sampled Functional Data. 2003; 98:397-408.
27. Luan Y, Li H. Clustering of time-course gene expression data using a mixed-effects model with B-splines. Bioinformatics (Oxford, England) 2003; 19:474-482.
28. Rice J A, Wu C O. Nonparametric mixed effects models for unequally sampled noisy curves. Biometrics 2001; 57:253-259.
29. Storey J D, Xiao W, Leek J T, Tompkins R G, Davis R W. Significance analysis of time course microarray experiments. Proceedings of the National Academy of Sciences of the United States of America 2005; 102: 12837-12842.
30. Friedman J H, Silverman B W. Flexible parsimonious smoothing and additive modeling (with discussion). Technometrics 1989; 3 1:3-39.
31. Gratwohl A, Hermans J, Apperley J, et al. Acute graft-versus-host disease: grade and outcome in patients with chronic myelogenous leukemia. Working Party Chronic Leukemia of the European Group for Blood and Marrow Transplantation. Blood 1995; 86:813-818.
32. James G M, Hastie T J. Functional linear discriminant analysis for irregularly sampled curves. Journal of the Royal Statistical Society Series B 2001; 63:533-550.
33. Cohen J L, Boyer 0. The role of CD4+CD25hi regulatory T cells in the physiopathogeny of graft-versus-host disease. Curr Opin Immunol 2006; 18:580-585.
34. Nguyen V H, Zeiser R, Negrin R S. Role of naturally arising regulatory T cells in hematopoietic cell transplantation. Biol Blood Marrow Transplant 2006; 12:995-1009.
35. Boeckh M, Fries B, Nichols W G. Recent advances in the prevention of CMV infection and disease after hematopoietic stem cell transplantation. Pediatr Transplant 2004; 8 Suppl 5:19-27.
36. Nascimbeni M, Shin E C, Chiriboga L, Kleiner D E, Rehermann B. Peripheral CD4(+)CD8(+) T cells are differentiated effector memory cells with antiviral functions. Blood 2004; 104:478-486.
37. Ortolani C, Forti E, Radin E, Cibin R, Cossarizza A. Cytofluorimetric identification of two populations of double positive (CD4+,CD8+) T lymphocytes in human peripheral blood. Biochem Biophys Res Commun 1993; 191:601-609.
38. Tonutti E, Sala P, Feruglio C, Yin Z, Colombatti A. Phenotypic heterogeneity of persistent expansions of CD4+CD8+ T cells. Clin Immunol Immunopathol 1994; 73:312-320.
39. Weiss L, Roux A, Garcia 5, et al. Persistent expansion, in a human immunodeficiency virus-infected person, of V beta-restricted CD4+CD8+ T lymphocytes that express cytotoxicity-associated molecules and are committed to produce interferon-gamma and tumor necrosis factor-alpha. J Infect Dis 1998; 178:1158-1162.
40. Jimenez E, Sacedon R, Vicente A, Hernandez-Lopez C, Zapata A G, Varas A. Rat peripheral CD4+CD8+ T lymphocytes are partially immunocompetent thymus-derived cells that undergo post-thymic maturation to become functionally mature CD4⁺T lymphocytes. J Immunol 2002; 168:5005-5013.
41. Pahar B, Lackner M, Veazey R S. Intestinal double-positive CD4+CD8+ T cells are highly activated memory cells with an increased capacity to produce cytokines. Eur J Immunol 2006; 36:583-592.
42. Szczepanik M, Bryniarski K, Tutaj M, et al. Epicutaneous immunization induces alphabeta T-cell receptor CD4 CD8 double-positive non-specific suppressor T cells that inhibit contact sensitivity via transforming growth factor-beta. Immunology 2005; 115:42-54.
43. Lambert C, Iobagiu C, Genin C. Persistent oligoclonal CD4^{dim}CD8+ T cells in peripheral blood. Cytometry B Clin Cytom 2005; 66:10-17.
44. Chen B J, Deoliveira D, Cui X, et al. Inability of memory T cells to induce graft-versus-host disease is a result of an abortive alloresponse. Blood 2006.

1. A method for diagnosing graft versus host disease (GvHD) or determining risk of developing GvHD in a susceptible subject comprising:
measuring CD3⁺CD4⁺CD8β⁺ cells in peripheral blood, and
comparing CD3⁺CD4⁺CD8β⁺ cells with a control value;
wherein an increase in CD3⁺CD4⁺CD8β⁺ cells in peripheral blood, a high level of CD3⁺CD4⁺CD8β⁺ cells in peripheral blood, or high fluctuation in CD3⁺CD4⁺CD8β⁺ cells in peripheral blood is indicative of GvHD or a risk of developing GvHD.

2. The method of claim 1 comprising periodic measurement of CD3⁺CD4⁺CD8β⁺ cells in peripheral blood.

3. The method of claim 2, wherein periodic measurement is conducted at an interval of from about 1 day to about 20 days.

4. The method of claim 3, wherein the interval is about 1 week.

5. The method of claim 2, wherein comparison with a control value comprises comparison of a subject's current measurement of CD3⁺CD4⁺CD8β⁺ cells with a subject's previous measurement.

6. The method of claim 5, wherein comparison with a control value comprises determining an increase in the subject's current measurement relative to a previous measurement, and comparison with a threshold increase above which GvHD or risk of developing GvHD is indicated.

7. The method of claim 1, wherein comparison with a control value comprises comparison of the subject's measurement of CD3⁺CD4⁺CD8β⁺ cells with a threshold value above which GvHD or risk of developing GvHD is indicated.

8. The method of claim 7, wherein the threshold value is determined from a healthy population, a population of transplant recipients without GvHD, or from a population of transplant recipients observed to subsequently develop GvHD.

9. The method of claim 2, wherein fluctuation in CD3⁺CD4⁺CD8β⁺ cells in peripheral blood is determined by comparing fluctuation in a subject's periodic measurements with a control value.

10. The method of claim 1, wherein measurement of CD3⁺CD4⁺CD8β⁺ cells in peripheral blood is conducted using flow cytometry.

11. The method of claim 1, wherein GvHD comprises acute GvHD.

12. The method of claim 1 additionally comprising assessing one or more parameter of immune function to diagnose or determine risk of developing GvHD, the parameter of immune function comprising: cytokine production, intracellular cytokines, measurement of T-regulatory cells, apoptosis status, cell cycle status, cellular activation phenotypes or signal transduction phenotypes.

13. A method of treating graft versus host disease (GvHD) comprising diagnosing GvHD or risk of developing GvHD in a susceptible subject according to the method of claim 1, and providing to a subject diagnosed with or at risk of developing GvHD an anti-GvHD therapeutic regime prior to observing clinical symptoms of GvHD.

14. The method according to claim 13, wherein the anti-GvHD therapeutic regime comprises administration of immunosuppressive medication.

15. The method of claim 14, wherein the immunosuppressive medication comprises a steroid, cyclosporine, a monoclonal antibody, Mycophenolate mofetil, Alemtuzumab, Antithymocyte globulin, FK506, Sirolimus, or a combination thereof.

16. The method of claim 13 additionally comprising assessing one or more parameter of immune function to diagnose or determine risk of developing GvHD, the parameter of immune function comprising: cytokine production, intracellular cytokines, measurement of T-regulatory cells, apoptosis status, cell cycle status, cellular activation phenotypes or signal transduction phenotypes.

17. The method of claim 13 wherein GvHD comprises acute GvHD.

18. A kit for diagnosing graft versus host disease (GvHD) or determining risk of developing GvHD in a susceptible subject comprising:
antibodies for detection of CD3⁺CD4⁺CD8β⁺ cells via flow cytometry analysis, and instructions for use.

19. (canceled)
